(19) United States
(12) Patent Application Publication
(10) Pub. No.: US 2023/0102924 A1
(43) Pub. Date: Mar. 30, 2023

(54) Methods of Treating Cancer Using Checkpoint Inhibitors in Combination with Purine Cleaving Enzymes

(71) Applicants: Emory University, Atlanta, GA (US); Children's Healthcare of Atlanta, Inc., Atlanta, GA (US)

(72) Inventors: Eric Sorscher, Atlanta, GA (US); Jeong Hong, Atlanta, GA (US); Turang Behbahani, Atlanta, GA (US); Regina Rab, Atlanta, GA (US); Annette Ehrhardt, Atlanta, GA (US); Disha Joshi, Atlanta, GA (US)

(21) Appl. No.: 17/908,465

(22) PCT Filed: Mar. 10, 2021
(86) PCT No.: PCT/US2021/021698

§ 371 Date: Aug. 31, 2022

Related U.S. Application Data
(60) Provisional application No. 62/987,780, filed on Mar. 10, 2020.

(51) Int. Cl. A61K 38/16; A61K 31/7076; A61P 35/00

(52) U.S. Cl. CPC A61K 38/164; A61K 31/7076; A61P 35/00

(57) ABSTRACT

This disclosure relates to methods of treating cancer or initiating, enhancing, or prolonging an anti-tumor response in a subject in need thereof comprising administering to the subject an effective amount of a checkpoint inhibitor in combination with a purine cleaving enzyme or a vector encoding expression thereof, and a prodrug cleaved by said purine cleaving enzyme. In certain embodiments, this disclosure relates to methods of treating cancer or initiating, enhancing, or prolonging an anti-tumor response in a subject in need thereof comprising administering to the subject an effective amount of a checkpoint inhibitor in combination with a purine cleaving enzyme, or a vector encoding expression thereof, in the absence of a prodrug cleaved by said purine cleaving enzyme.

### CROSS-REFERENCE TO RELATED APPLICATIONS

This application claims the benefit of U.S. Provisional Application No. 62/987,780 filed Mar. 10, 2020. The entirety of this application is hereby incorporated by reference for all purposes.

### BACKGROUND

Tumor cells evade the immune system by altering immune checkpoint pathways that suppress antitumor responses. Immune checkpoint inhibitors interrupt co-inhibitory signaling pathways and promote immune targeting of tumor cells. However, only a fraction of cancer patients benefits from checkpoint inhibitors, and immune-related adverse events are seen in some patients. Thus, there is a need to identity improved therapies.

Intratumoral injection of a vector for expression of a gene encoding an enzyme specific for a prodrug substrate followed by systemic prodrug treatment can be used to generate a toxic agent within a malignant mass. Tumor-directed expression of cytosine deaminase (CD), for example, has been applied to produce 5-fluorouracil (5-FU) from 5-fluorocytosine. F-Ade (2-fluoroadenine) disrupts crucial pathways required for cell viability. Intratumoral production of F-Ade elicits pronounced tumor involution in vivo. Rosenthal et al. report phase I dose-escalating trial of Escherichia coli purine nucleoside phosphorylase and fludarabine gene therapy for advanced solid tumors. Ann Oncol, 2005, 26(7): 1481-1487. Behbahani et al. report intratumoral generation of 2-fluoroadenine to treat solid malignancies of the head and neck. Head Neck, 2019, 41(6):1979-1983. Parker et al. report the use of E. coli purine nucleoside phosphorylase (PNP) in the treatment of solid tumors. Curr Pharm Des. 2018, 23(45):7003-7024. Parker et al. report the use of Trichomonas vaginalis purine nucleoside phosphorylase to activate fludarabine in the treatment of solid tumors. Cancer Chemother Pharmacol. 2020, 85(3):573-583.

References cited herein are not an admission of prior art.

### SUMMARY

This disclosure relates to methods of treating cancer or initiating, enhancing, or prolonging an anti-tumor response in a subject in need thereof comprising administering to the subject an effective amount of a checkpoint inhibitor in combination with a purine cleaving enzyme or a vector encoding expression thereof, and a prodrug cleaved by said purine cleaving enzyme. In certain embodiment, the purine cleaving enzyme is a non-mammalian purine nucleoside phosphorylase (PNP) or nucleoside hydrolase (NH).

In certain embodiment, the prodrug is 9-(β-D-arabinofuranosyl)-2-fluoroadenine (F-araA), 2-F-2′-deoxyadenosine (F-dAdo), fludarabine phosphate (F-araAMP, 2-fluoro-9-(5-O-phosphono-β-D-arabinofuranosyl)-9H-purin-6-amine), derivative, or salt thereof.

In certain embodiment, checkpoint inhibitor is a biologic therapeutic or a small molecule. In certain embodiment, checkpoint inhibitor is a monoclonal antibody, a humanized antibody, a fully human antibody, a fusion protein or a combination thereof. In certain embodiment, checkpoint inhibitor is a PD-1, a PDL-1 and/or a CTLA-4 checkpoint inhibitor. In certain embodiment, checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof.

In certain embodiment, checkpoint inhibitor is selected from ipilimumab (anti-CTLA-4 antibody), nivolumab, pembrolizumab, and cemiplimab (anti-PD-1 antibodies), atezolizumab, durvalumab, and avelumab (PD-L1).

In certain embodiments, this disclosure relates to methods of treating cancer or initiating, enhancing, or prolonging an anti-tumor response in a subject in need thereof comprising administering to the subject an effective amount of a checkpoint inhibitor in combination with a purine nucleoside phosphorylase or nucleoside hydrolase or a vector encoding expression thereof in the absence of a prodrug cleaved by said purine cleaving enzyme.

In certain embodiments, disclosure relates to methods of treating cancer or initiating, enhancing, or prolonging an anti-tumor response in a subject in need thereof comprising administering to the subject an effective amount of a checkpoint inhibitor in combination with and a prodrug in the absence of a purine cleaving enzyme or a vector encoding expression thereof capable of cleavage by said purine cleaving enzyme.

In certain embodiment, administering to the subject a checkpoint inhibitor in combination with a purine nucleoside phosphorylase or nucleoside hydrolase or a vector encoding expression thereof is a direct injection of the purine nucleoside phosphorylase or nucleoside hydrolase or a vector encoding expression thereof into replicating or non-replicating targeted cells and optionally exposure of the targeted cells to X-ray radiation. In certain embodiment, the said replicating or non-replicating targeted cells are cancerous or define a tumor. In certain embodiment, the said viral vector is adenoviral vector or lentiviral vector. In certain embodiment, purine nucleoside phosphorylase is derived from E. coli or T. vaginalis or other bacterial strains. In certain embodiment, purine nucleoside phosphorylase is a mutant of E. coli PNP.

In certain embodiments, this disclosure relates to intratumoral injection of a vector for expression of E. coli PNP followed by systemic prodrug treatment. In certain embodiment, this disclosure relates to the use of intratumoral expression of E. coli PNP or other non-mammalian proteins to increase anti-cancer activity of immune-type therapeutic agents. In certain embodiments, immune-type therapies include checkpoint blockade inhibitors including CTLA-4 blockers, PD1 antibodies, PD1 ligand antibodies, or T- or B-cell therapies.

In certain embodiment, this disclosure relates to the use of intratumoral expression of PNP or other non-mammalian proteins to produce an abscopal effect (i.e. distant tumors not expressing PNP or other non-mammalian proteins exhibit blunted growth as a result of PNP/other protein expression in a single tumor) in the setting of immune-type therapies.

In certain embodiment, this disclosure relates to the use of a checkpoint inhibitor in combination with intratumoral PNP expression followed by nucleoside-mediated tumor regression (or other gene therapy-based molecular chemotherapies) as a means to provide synergy with checkpoint blockade-type agents.

In certain embodiment, this disclosure relates to the use of a checkpoint inhibitor in combination with intratumoral PNP (or expression of other PNPs or other nucleoside-metabolizing enzymes) together with nucleoside-mediated tumor regression to provide an abscopal effect and blunt growth of distant tumors not expressing the therapeutic transgene in the setting of immune type therapies.

In certain embodiment, this disclosure relates to the use of targeted destruction of a subset of tumors with chemotherapy (including PNP-based treatments, other gene-based molecular chemotherapies, and intratumoral or systemic administration of chemotherapy) to enhance checkpoint blockade or other immune-type treatments against additional tumors in the same host. In certain embodiments, the PNP-based treatments are E. coli PNP and fludarabine phosphate or T. vaginalis PNP and fludarabine phosphate.

In certain embodiment, this disclosure relates to the use of fludarabine phosphate or other immune modulator drugs including compounds that inhibit the immune system to provide enhancement of checkpoint blockade or other immune-type therapies.

In certain embodiment, this disclosure relates to sensitizing hematologic malignancies to checkpoint blockade in which a small subset of the malignant cell population expresses non-mammalian proteins such as E. coli PNP (with or without fludarabine phosphate treatment) so that the entire tumor cell burden becomes more sensitive to immune-type therapies or drugs such as fludarabine phosphate.

In certain embodiments, this disclosure contemplates using fludarabine phosphate or fludarabine phosphate derivatives, other small molecules nucleosides, or nucleoside derivatives which have the same activity, as a way to augment checkpoint blockage by impacting T cells and other immune processes, e.g., fludarabine phosphate or derivative as a single agent.

In certain embodiments, this disclosure contemplates PNP expression in tumor parenchyma to alert the immune system and enhance the checkpoint blockade optionally in the presence or absence of a prodrug or derivative. In certain embodiments, this disclosure contemplates other prokaryotic protein expression in tumor parenchyma to alert the immune system and enhance the checkpoint blockade optionally in the presence of absence of a prodrug.

### BRIEF DESCRIPTION OF THE SEVERAL VIEWS OF THE DRAWINGS

FIG. 1 shows data from experiments using groups 1, 2, 4 & 5 mice, as described in table 1 of the examples, to determine the effect of fludarabine phosphate (F-araAMP, 75 mg/kg) plus or minus anti-CTLA4-9H10 on EMT-6 breast tumors that express E. coli PNP. Tumors on left flank express E. coli PNP (in groups 1-6). The data indicates PNP/fludarabine phosphate (75 mg/kg) and anti-CTLA4 antibody are effective against murine breast tumors.

FIG. 2 shows data from experiments using groups 1, 3, 4 & 6 mice, as described in table 1, in order to determine the effect of fludarabine phosphate (F-araAMP, higher dose 90 mg/kg) plus or minus anti-CTLA4-9H10 on EMT-6 tumors that express E. coli PNP. Tumors on left flank express E. coli PNP. Group 4 mice are significantly different than group 6 mice on day 15, and group 3 mice are significantly different than group 6 mice on day 15 indicating PNP/fludarabine phosphate (90 mg/kg) and anti-CTLA4 antibody are effective against murine breast tumors.

FIG. 3 shows data from experiments using groups 1, 3, 4 & 6 mice, as described in table 1, in order to determine the effect of fludarabine phosphate (F-araAMP, 90 mg/kg) plus or minus anti-CTLA4-9H10 on EMT-6 tumors that do not express E. coli PNP. None of the tumors on right flank express E. coli PNP. Group 4 mice are significantly different than group 6 mice (day 15), if regressed tumors are removed from the analysis; however, group 4 mice are not significantly different than group 6 mice (day 15), if all tumors are included in the analysis, indicating anti-CTLA4 antitumor activity is augmented by fludarabine phosphate as a single agent when a PNP-tumor is regressing due to PNP/fludarabine phosphate (FIG. 3) contralaterally (i.e., abscopal effect).

FIG. 4 shows data from experiments using groups 7, 8, 9 & 10 mice, as described in table 1, in order to determine the effect of fludarabine phosphate (F-araAMP, 90 mg/kg) plus or minus anti-CTLA4-9H10 on EMT-6 tumors that do not express E. coli PNP. Group 9 mice are significantly different than group 10 mice (day 1) indicating fludarabine phosphate (90 mg/kg) augments anti-CTLA4 antibody activity in parental (non-PNP) tumors.

FIG. 5 shows data from experiments using groups 1, 3, 4 & 6 mice, as described in table 1, in order to determine the effect of fludarabine phosphate (F-araAMP, 90 mg/kg) plus or minus anti-CTLA4-9H10 on EMT-6 tumors that do not express E. coli PNP. Group 4 mice are significantly different than group 6 (day 15) mice, if regressed tumors are removed from the analysis; however, group 4 are not significantly different than group 6 (day 15), if all tumors are included in the analysis indicating fludarabine phosphate (90 mg/kg) augments anti-CTLA4 antibody activity in parental (non-PNP) tumors.

FIG. 6 shows data from experiments using groups 7, 8, 9 & 10 mice, as described in table 1, in order to determine the effect of fludarabine phosphate (F-araAMP, 90 mg/kg) plus or minus anti-CTLA4-9H10 on EMT-6 tumors that do not express E. coli PNP. Group 9 are mice significantly different than group 10 mice (day 15) indicating fludarabine phosphate (90 mg/kg) augments anti-CTLA4 antibody activity in parental (non-PNP) tumors.

FIG. 7 shows data from experiments using groups 1, 6, and 10 mice, as described in table 1, in order to determine the effect of fludarabine phosphate (F-araAMP, 90 mg/kg) plus or minus anti-CTLA4-9H10 on EMT-6 tumors. In group 6, the right flanks were implanted with parental (non-PNP) tumors and the left flanks were implanted with tumors expressing E. coli PNP. Group 10 mice did not express PNP in tumors on neither left nor right flanks. Group 6 mice are significantly different than either group 10 mice (right) or group 10 mice (left) (day 15). These results indicate a PNP tumor on the left flank, which is regressing due to F-araAMP, causes an abscopal effect on a non-PNP tumor, contralaterally.

FIG. 8 shows data from experiments using groups 1, 4, 7, & 9 mice, as described in table 1, in order to determine the effect of anti-CTLA4-9H10 on EMT-6 tumors that express E. coli PNP or that do not express E. coli PNP. Tumors in open symbols express E. coli PNP. Tumors in closed symbols do not express E. coli PNP. Group 7 mice are not significantly different than group 9 (day 15). Group 4 mice are also significantly different than group 1 mice (day 15). Group 4 mice are significantly different than group 9 mice (day 15); however, if regressed tumors are removed from the analysis, group 4 is not significantly different than group 9 mice (day 15). These results suggest tumors expressing E. coli PNP are sensitized to anti-CTLA4 antibody.

FIG. 9 shows data from experiments using groups 1 & 4 mice (right and left), as described in table 1, in order to determine the effect of anti-CTLA4-9H10 on EMT-6 tumors that express E. coli PNP or that do not express E. coli PNP. Closed symbols indicate the use of parental tumors on the right flank. Open symbols indicate the use of tumors on left flank that express E. coli PNP. Group 4 (right flank) tumors are not significantly different than group 4 tumors (left flank) (day 15)(not including regressed tumors) suggesting tumors expressing PNP are not more sensitive to anti-CTLA4 antibody.

FIG. 10 shows experiments to evaluate the abscopal effect of PNP expression without fludarabine phosphate in the presence of anti-CTLA4 antibody. Groups 1, 4, 7, & 9, as described in table 1, in order to determine the effect of anti-CTLA4-9H10 on EMT-6 tumors that do not express E. coli PNP. Tumors on right flank are shown. None of these tumors express E. coli PNP. Open symbols indicate the use of contralateral tumors that express E. coli PNP. Closed symbols indicate the use of contralateral tumors that do not express E. coli PNP. Growth of a non-PNP tumor (on an animal carrying a PNP tumor contralaterally) in the presence of anti-CTLA4 antibody resulted in the regression of 2 of 6 tumors, whereas there were 0 of 6 regressions seen in non-PNP tumors in an animal with a non-PNP tumor contralaterally. The growth of the remaining 4 non-PNP tumors (on an animal carrying a PNP tumor contralaterally) was not different than that of the 6 non-PNP tumors (on an animal carrying a non-PNP tumor contralaterally).

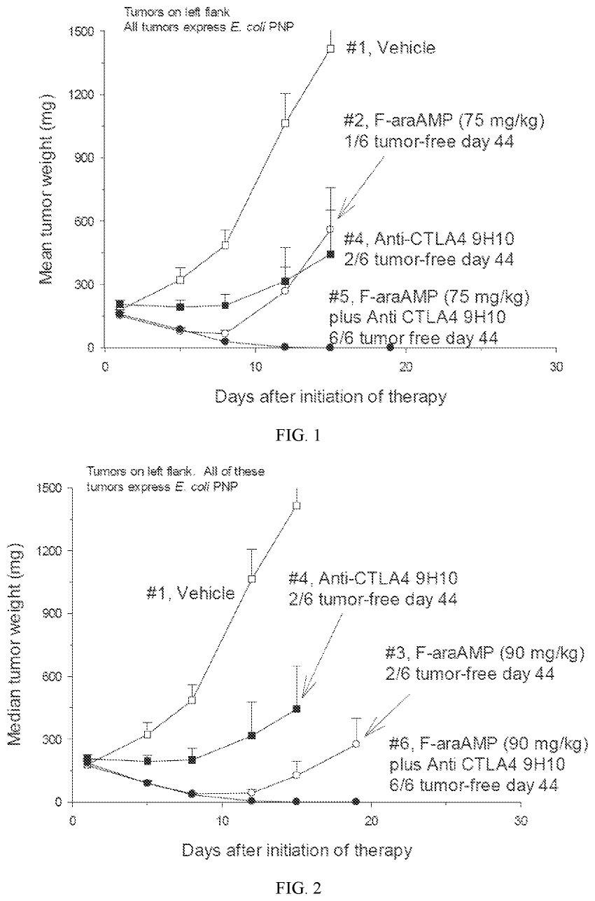

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, rodent, monkey or domestic pet.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof. As used herein, the term “intermixed with” when used to describe administration in combination with an additional treatment means that the agent may be administered “together with.”

The term “effective amount” refers to that amount of a compound or pharmaceutical composition described herein that is sufficient to effect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

Combination Therapies with Checkpoint Inhibitors, Purine Cleaving Enzymes, Prodrugs

In certain embodiments, this disclosure relates to methods of treating cancer or initiating, enhancing, or prolonging an anti-tumor response in a subject in need thereof comprising administering to the subject an effective amount of a checkpoint inhibitor in combination with a purine cleaving enzyme or a vector encoding expression thereof, and a prodrug cleaved by said purine cleaving enzyme.

In certain embodiment, this disclosure relates to methods of enhancing or prolonging the effects of a checkpoint inhibitor, or enabling a subject to respond to a checkpoint inhibitor, or enabling the toxicity or the dose or number of treatments of a checkpoint inhibitor to be reduced, comprising administering to a subject in need thereof a purine nucleoside phosphorylase or nucleoside hydrolase in combination with or intermixed with a prodrug in combination with a checkpoint inhibitor.

In certain embodiment, this disclosure relates to methods of enhancing or prolonging the effects of a checkpoint inhibitor, or enabling a subject to respond to a checkpoint inhibitor, or enabling the toxicity or the dose or number of treatments of a checkpoint inhibitor to be reduced, comprising administering to a subject in need thereof a purine nucleoside phosphorylase or nucleoside hydrolase in combination with a checkpoint inhibitor in the absence of a prodrug.

In certain embodiment, this disclosure relates to methods of enhancing or prolonging the effects of a checkpoint inhibitor, or enabling a subject to respond to a checkpoint inhibitor, or enabling the toxicity or the dose or number of treatments of a checkpoint inhibitor to be reduced, comprising administering to a subject in need thereof a prodrug in combination with a checkpoint inhibitor in the absence of a purine nucleoside phosphorylase or nucleoside hydrolase.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, checkpoint inhibitor interacts with a ligand of a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In certain embodiments, the checkpoint inhibitor is an anti-CTLA-4 antibody such as ipilimumab, an anti-PD-1 antibody such as pembrolizumab, nivolumab, REGN2810, BMS-936558, SHR1210, IBI308, PDR001, BGB-A317, BCD-100, and JS001 or an anti-PD-L1 antibody such as avelumab, atezolizumab, durvalumab, and KN035. In certain embodiments, administering to the subject an effective amount of a checkpoint inhibitor is a combination of an anti-CTLA-4 antibody and an anti-PD-1 antibody.

In certain embodiments, the checkpoint inhibitor and the purine nucleoside phosphorylase or nucleoside hydrolase or vector encoding expression thereof in combination with or intermixed with a prodrug are administered simultaneously or sequentially, in either order. In an additional aspect, the purine nucleoside phosphorylase or nucleoside hydrolase or vector encoding expression thereof in combination with or intermixed with a prodrug is administered prior to the checkpoint inhibitor. In a specific aspect, the purine nucleoside phosphorylase or nucleoside hydrolase or vector encoding expression thereof in combination with or intermixed with a prodrug and the checkpoint inhibitor is a PD-1 or a PDL-1 or CTLA-4 inhibitor.

In certain embodiments, the subject has cancer. In another aspect, the cancer is any solid tumor or liquid cancers, including urogenital cancers (such as prostate cancer, renal cell cancers, bladder cancers), gynecological cancers (such as ovarian cancers, cervical cancers, endometrial cancers), lung cancer, gastrointestinal cancers (such as non-metastatic or metastatic colorectal cancers, pancreatic cancer, gastric cancer, esophageal cancers, hepatocellular cancers, cholangiocellular cancers), head and neck cancer (e.g. head and neck squamous cell cancer), brain cancers including malignant gliomas and brain metastases, malignant mesothelioma, non-metastatic or metastatic breast cancer (e.g. hormone refractory metastatic breast cancer), malignant melanoma, Merkel Cell Carcinoma or bone and soft tissue sarcomas, and hematologic neoplasias, such as multiple myeloma, acute myelogenous leukemia, chronic myelogenous leukemia, myelodysplastic syndrome and acute lymphoblastic leukemia. In a preferred embodiment, the disease is non-small cell lung cancer (NSCLC), breast cancer (e.g. hormone refractory metastatic breast cancer), head and neck cancer (e.g. head and neck squamous cell cancer), metastatic colorectal cancers, hormone sensitive or hormone refractory prostate cancer, colorectal cancer, ovarian cancer, hepatocellular cancer, renal cell cancer, soft tissue sarcoma, or small cell lung cancer.

In certain embodiments, methods disclosed herein further comprise administering the combination of agents disclosed herein or radiation to the subject either prior to, simultaneously with, or after treatment with the combination therapy. In an additional aspect, the tumor may be resected prior to the administration of the purine nucleoside phosphorylase or nucleoside hydrolase or vector encoding expression thereof in combination with or intermixed with a prodrug and checkpoint inhibitor.

In a further embodiment, the disclosure provides for a pharmaceutical composition comprising a checkpoint inhibitor in combination with a purine nucleoside phosphorylase or nucleoside hydrolase or vector encoding expression thereof.

In a further embodiment, the disclosure provides for a pharmaceutical composition comprising a checkpoint inhibitor in combination with a prodrug degraded by a purine nucleoside phosphorylase or nucleoside hydrolase.

In certain embodiments, the pharmaceutical composition is in the form of a tablet, pill, capsule, gel, gel capsule, or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutical composition is in solid form surrounded by an enteric coating. In certain embodiments, the enteric coatings comprises a component such as methyl acrylate-methacrylic acid copolymers, cellulose acetate phthalate (CAP), cellulose acetate succinate, hypromellose (hydroxypropyl methylcellulose), hypromellose phthalate (hydroxypropyl methyl cellulose phthalate), hypromellose acetate succinate (hydroxypropyl methyl cellulose acetate succinate), diethyl phthalate, polyvinyl acetate phthalate (PVAP), methyl methacrylate-methacrylic acid copolymers, or combinations thereof.

In certain embodiments, the pharmaceutically acceptable excipient is selected from lactose, sucrose, mannitol, triethyl citrate, dextrose, cellulose, microcrystalline cellulose, methyl cellulose, ethyl cellulose, hydroxyl propyl cellulose, hydroxypropyl methylcellulose, carboxymethylcellulose, croscarmellose sodium, polyvinyl N-pyrrolidone (crospovidone), ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In a further aspect, the anti-tumor response is inhibiting tumor growth, inducing tumor cell death, tumor regression, preventing or delaying tumor recurrence, tumor growth, tumor spread or tumor elimination.

In one embodiment, the present disclosure provides for a method for the combination therapy for the treatment of cancer wherein the combination therapy comprises (a) purine nucleoside phosphorylase or nucleoside hydrolase or vector encoding expression thereof in combination with or intermixed with a prodrug and (b) a checkpoint inhibitor.

In another embodiment, the present disclosure provides for a method for initiating, sustaining or enhancing an anti-tumor immune response, the method comprising administering to a subject (a) a purine nucleoside phosphorylase or nucleoside hydrolase or vector encoding expression thereof in combination with or intermixed with a prodrug and (b) a checkpoint inhibitor. In specific aspects, the purine nucleoside phosphorylase or nucleoside hydrolase or vector encoding expression thereof in combination with or intermixed with a prodrug is administered before the checkpoint inhibitor. In specific embodiments, the purine nucleoside phosphorylase or nucleoside hydrolase or vector encoding expression thereof in combination with or intermixed with a prodrug is administered immediately, up to 1 hour, up to 2 hours, up to 3 hours, up to 4 hours, up to 5 hours, up to 6 hours or up to 1-30 days before or after the checkpoint inhibitor. In specific aspects, the anti-tumor response is a tumor specific response, a clinical response, a decrease in tumor size, stabilization of a tumor, a decrease in tumor specific biomarkers, increased tetramer staining, an increase in anti-tumor or pro-inflammatory cytokines or a combination thereof. In a specific aspect, the clinical response is a decreased tumor growth and/or a decrease in tumor size. In a specific aspect, the initiating, sustaining or enhancing an anti-tumor immune response is for the treatment of cancer.

In a further embodiment, the present disclosure provides a method for enhancing the efficacy of a checkpoint inhibitor, or enabling a subject to respond to a checkpoint inhibitor, the method comprising administering to a subject (a) a purine nucleoside phosphorylase or nucleoside hydrolase or vector encoding expression thereof in combination with or intermixed with a prodrug (b) a checkpoint inhibitor. In specific aspects, at least 30%, 40%, 50%, 60%, 70%, 80%, or 90% of subjects respond to the administration of a purine nucleoside phosphorylase or nucleoside hydrolase or vector encoding expression thereof in combination with or intermixed with a prodrug and a checkpoint inhibitor.

In a further embodiment, the checkpoint inhibitor described herein may comprise one or more separate checkpoint inhibitors. Moreover, the administration of (a) a purine nucleoside phosphorylase or nucleoside hydrolase or vector encoding expression thereof is in combination with or intermixed with a prodrug and (b) a checkpoint inhibitor described herein may reduce an effective amount of checkpoint inhibitor to be administered to a subject or patient. Further, the reduced amount of the checkpoint inhibitor may reduce the toxicity of the checkpoint inhibitor and increase the tolerance of the subject to the checkpoint inhibitor.

A purine cleaving enzyme may be a purine nucleoside phosphorylase (PNP) or nucleoside hydrolase (NH) such as that obtained from E. coli, Trichomonas vaginalis, or any other nonhuman PNP which can convert a prodrug substrate to produce a cytotoxic purine base. Non-host PNPs or nucleoside hydrolases along with a suitable prodrug are appreciated to also be operative herein as a basis to practice the present disclosure. The prodrug, through specific cleavage, is selected to produce a comparatively higher cytotoxicity compound. It is further appreciated that mutant PNPs and hydrolases such as those detailed in U.S. Pat. No. 7,488,598 are operative herein to generate a cytotoxic purine base from the prodrug and suitable for inhibiting cellular function such as reproduction and even killing of those cells of a human subject that have been transfected or are simply in proximity to the enzyme. It is appreciated that an enzyme as used herein may afford a cytotoxic purine base of sufficient potency to generate a bystander effect thereby inhibiting transfected cells, transduced cells, as well as bystander cells.

As used herein “proximity” is intended to mean introduction directly into a defined tissue mass, such as for example a tumor mass, as well as adjacent to a target cell within a spacing of, for example, approximately or less than 50 or 20 adjacent cell diameters or equivalent linear spacing and preferably within 20 adjacent cell diameters or equivalent linear spacing.

A prodrug operative herein has the attribute of being relatively nontoxic to subject cells yet upon enzymatic cleavage of the prodrug produces a cytotoxic purine base. In certain embodiments the prodrug is selected from 2-F-2′-deoxyadenosine (F-dAdo) or fludarabine phosphate (F-araAMP). Other examples include allo-met: 9-(6-deoxy-β-D-allofuranosyl)-6-methylpurine; talo-met: 9-(6-deoxy-α-L-talofuranosyl)-6-methylpurine; 5′-NH2: 5′-amino-5′-deoxyadenosine; allo-acet: 9-(6,7-dideoxy-β-D-hept-6-ynofuranosyl)-6-methylpurine; talo-acet: 9-(6,7-dideoxy-α-L-hept-6-ynofuranosyl)-6-methylpurine; α-L-lyxo: 9-(α-L-lyxofuranosyl)-adenine; 5′-CONH2: adenosine 5′-carboxamide; 5′-S-phenyl: 9-(5-deoxy-5-phenylthio-β-D-ribofuranosyl)-6-methylpurine; MeP-dR: 9-(2-deoxy-β-D-ribofuranosyl)-6-methylpurine; F-araA: 9-(β-D-arabinofuranosyl)-2-fluoroadenine; 5′-methyl(allo)-MeP-R: 9-(6-deoxy-β-D-allofuranosyl)-6-methylpurine; 5′-methy(talo)-MeP-R: 9-(6-deoxy-α-L-talofuranosyl)-6-methylpurine; F-Ade: 2-fluoroadenine; and 5′-methyl(talo)-2-F-adenosine: 9-(6-deoxy-α-L-talofuranosyl) fluoroadenine; or combinations thereof.

In certain embodiments, this disclosure relates to a process for generating a very potent cytotoxic agent specifically within a target cell volume in general and specifically in tumor parenchyma. The limited radius of F-Ade diffusion following generation within a tumor mass and extensive dilution (to unmeasurable F-Ade levels in serum) after release from dying tumor cells and confers consistent in vivo bystander killing with manageable host toxicity.

In certain embodiments, the amount of the prodrug, e.g., F-araAMP routinely administered as part of a therapy in humans is about 25 mg/m² per dose×5 daily doses given every 4 weeks. The present disclosure contemplates a therapeutic modality in which Ad/PNP followed by F-araAMP are administered repeatedly to needle-accessible tumors (prostate, breast, head and neck, or with radiology guidance, other tumor masses) on a frequent (e.g., daily) basis to sequentially destroy large regions of a tumor while minimizing systemic exposure to either F-araAMP, F-Ade, or other PNP cleaved prodrug. A “point and ablate” approach is feasible because of the potent antitumor activity of F-Ade and its high bystander activity, together with activity against nonproliferating tumor cells. Intratumoral generation of F-Ade should provide a means to concentrate the agent intratumorally and minimize systemic exposure in the host.

In the method described above, the mammalian cells to be killed can be tumor cells. Cells comprising any solid tumor, whether malignant or not, can be killed by the present method based on the ability to transfer or express the PNP or NH gene selectively to at least a small percentage of cells comprising the tumor. For example, it has been shown that intravenous injection of liposome carrying DNA can mediate targeted expression of genes in certain cell types.

In addition to killing tumor cells, methods of this disclosure can also kill virally infected cells. In a virus-killing embodiment, the gene transfer method selected would be chosen for its ability to target the expression of PNP in virally infected cells. For example, virally infected cells may utilize special viral gene sequences to regulate and permit gene expression (i.e., virus specific promoters). Such sequences are not present in uninfected cells. In certain embodiments, it is contemplated that E. coli PNP or other PNP genes are oriented appropriately with regard to such a viral promoter, PNP would only be activated within virally infected cells, and no other, uninfected, cells. In this case, virally infected cells would be much more susceptible to the administration of substrates designed to be converted to toxic form by PNP or NH when delivered in proximity to target cells.

In other applications of the present disclosure, a medicament is provided to kill or otherwise inhibit the function of any desired target cell volume of a subject. The broad applicability to kill or otherwise inhibit function of cells affords clinical practitioners with control of administration, as well as improves healing profiles over a variety of conventional procedures. The present disclosure contemplates a chemical cellular ablation alternative to procedures involving cautery or excision. The chemical cellular ablation afforded by the present disclosure precludes the granulation and scarification associated with cautery, radioablation, or excision techniques thereby providing a superior healed tissue around the situs of chemical ablation and as a result, the present disclosure contemplates the treatment of cardiac arrhythmia, cyst reduction, ganglion treatment, male sterilization, cosmetic dermatological procedures, and melanoma treatment. It is appreciated that chemical cellular ablation is readily performed by administration of PNP or NH enzyme, genes expressing any form of a viral vector as detailed herein; along with proximal delivery of a prodrug for the PNP or NH. Based on the location of the target cells for chemical cellular ablation, medicament is administered via a catheter, canula, or syringe; as well as topically in a cream base. Preferably, the PNP or NH enzyme is expressed intracellularly.

An isolated nucleic acid encoding a non-human or genetically modified human purine nucleoside phosphorylase or nucleoside hydrolase in a mammalian cell is contemplated. In certain embodiments, an isolated nucleic acid encoding an E. coli PNP in a mammalian cell is contemplated. By “isolated” is meant separated from other nucleic acids found in the naturally occurring organism from which the PNP gene is obtained.

A eukaryotic transfer vector comprising a nucleic acid encoding a non-human or genetically modified purine nucleoside phosphorylase or nucleoside hydrolase is also provided. The vector must be capable of transducing or transfecting at least some percentage of the cells targeted. The transfer vector can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, 1993). In certain embodiments, a lentiviral or adenoviral vector containing a nucleic acid encoding PNP are contemplated.

The vector can be in a host capable of expressing a functional PNP or NH. As used in the methods disclosed herein, the host cell is the cell to be killed, which expresses the PNP or NH and is killed by the toxic product of the reaction of the enzyme and the prodrug that is an enzymatic substrate.

In addition to the present gene transfer methods, the PNP gene product can also be selectively delivered to the tumor cells by a number of different mechanisms and this PNP could be used to produce F-Ade at the site of the tumor. For instance, the PNP or NH enzyme can be attached to any desired monoclonal antibody and injected into the patient either systemically or into proximity to target cells. After allowing sufficient time for the clearance of all PNP or NH conjugated to monoclonal antibody that has not bound to the target cells, the patient is treated by direct injection of the prodrug, such as F-araAMP, which is cleaved to F-Ade only at the targeted site. Such a procedure requires only the availability of an appropriate monoclonal antibody. The procedures used for conjugating proteins to target-specific monoclonal antibodies are routinely available. Other ligands, in addition to monoclonal antibodies, can be selected for their specificity for a target cell and tested according to the methods taught herein.

It is also possible to entrap proteins in liposomes and target them to specific tissues. The PNP or NH gene product can, thus, be selectively delivered to a tumor mass using targeted liposomes. After all non-targeted liposome is cleared from the blood, the patient is treated with F-araAMP which is cleaved to F-Ade by the PNP only at the targeted site. Once again, this procedure requires only the availability of an appropriate targeting vehicle.

A prodrug that represents enzymatic substrate for a non-host PNP or NH is injected directly into target cell mass as for example, intratumorally in a pharmaceutically acceptable carrier such as, for example, saline or DMSO, or alternatively, is encapsulated to modify prodrug stability and/or therapeutic characteristics. A prodrug is readily administered as a gel, paste or capsulated within microparticles. It is appreciated that such carriers for prodrugs are readily used to provide a prolonged release of the prodrug, modified diffusion within the targeted cell mass, and storage stability as compared to dissolution in a saline solution. With resort to microparticles, release rates of an inventive prodrug are readily extended to more than one week, more than two weeks, even beyond six weeks. A prodrug is readily prepared and injected in a paste of polylactic acid, poly(epsilon-caprolactone), or a combination thereof (Jackson et al., Cancer research 60 (15): 4146-4151, 2000). Prodrugs are also suitably encapsulated within microspheres from a variety of materials including polylactic acid, poly(epsilon-caprolactone), polyvinyl pyrrolidone, hydroxypropylcellulose, methyl cellulose, and other polysaccharides (Harper et al, Clin. Cane. Res. 5:4242-4248, 1999; Dordunno et al, Cancer Chemother. Pharmacol. 36: 279-282, 1995; Bert et al, Cancer Lett. 88:73-78, 1995) It is appreciated that with a controlled release formulation of prodrug, larger doses of prodrug are injected into a target cell mass less frequently to achieve a prolonged cell inhibition and bystander effect.

In certain embodiments, this disclosure contemplates use of a purine nucleoside phosphorylase or nucleoside hydrolase or a vector encoding expression thereof, and a prodrug cleaved by said purine nucleoside phosphorylase or nucleoside hydrolase for the preparation of a direct injection medicament for the functional inhibition or killing of replicating or non-replicating targeted cells. In certain embodiments, said purine nucleoside phosphorylase or nucleoside hydrolase is in combination with or intermixed with said prodrug. In certain embodiments, said prodrug is formulated with a sustained release carrier.

In certain embodiments, said purine nucleoside phosphorylase or nucleoside hydrolase is delivered with a viral vector containing a nucleic acid encoding said purine nucleoside phosphorylase or said nucleoside hydrolase. In certain embodiments, said viral vector is adenoviral vector.

In certain embodiments, said purine nucleoside phosphorylase is derived from E. coli or T. vaginalis. In certain embodiments, said purine nucleoside phosphorylase is a mutant of E. coli PNP. In certain embodiments, said mutant is a tailed mutant.

In certain embodiments, said prodrug is fludarabine phosphate. In certain embodiments, said replicating or non-replicating targeted cells are cancerous. In certain embodiments, substances of a purine nucleoside phosphorylase or nucleoside hydrolase or a vector encoding expression thereof and a prodrug cleaved by said purine nucleoside phosphorylase or nucleoside hydrolase are used with direct prodrug injection and inhibition of replicating or non-replicating targeted cells or targeted cells define a tumor.

In certain embodiments, this disclosure contemplates a process of inhibiting (replicating or non-replicating) targeted cells comprising: administering a check point inhibitor in combination with delivering a purine nucleoside phosphorylase or nucleoside hydrolase to the targeted cells defining a tumor; administering a prodrug cleaved by said purine nucleoside phosphorylase or nucleoside hydrolase to release a purine base cytotoxic to the targeted cells.

In certain embodiments, said prodrug is administered by intratumoral injection into said tumor. In certain embodiments, said purine nucleoside phosphorylase or nucleoside hydrolase is delivered with a viral vector containing a nucleic acid encoding said purine nucleoside phosphorylase or said nucleoside hydrolase.

In certain embodiments, treatment is determined by a clinical outcome; an increase, enhancement or prolongation of anti-tumor activity by T cells; an increase in the number of anti-tumor T cells or activated T cells as compared with the number prior to treatment or a combination thereof. In certain embodiments, clinical outcome is tumor stabilization, tumor regression or stabilization; tumor shrinkage; tumor necrosis; anti-tumor response by the immune system; inhibition of tumor expansion, recurrence or spread or a combination thereof. In certain embodiments, the treatment effect is predicted by presence and/or status of T cells, presence of a gene signature indicating T cell infiltration or inflammation or a combination thereof.

In certain embodiments, the subject has or is diagnosed with cancer. In an additional aspect, the cancer is any solid tumor or liquid cancers, including urogenital cancers (such as prostate cancer, renal cell cancers, bladder cancers), gynecological cancers (such as ovarian cancers, cervical cancers, endometrial cancers), lung cancer, gastrointestinal cancers (such as non-metastatic or metastatic colorectal cancers, pancreatic cancer, gastric cancer, esophageal cancers, hepatocellular cancers, cholangiocellular cancers), head and neck cancer (e.g. head and neck squamous cell cancer), brain cancers including malignant gliomas and brain metastases, malignant mesothelioma, non-metastatic or metastatic breast cancer (e.g. hormone refractory metastatic breast cancer), malignant melanoma, Merkel Cell Carcinoma or bone and soft tissue sarcomas, and hematologic neoplasias, such as multiple myeloma, acute myelogenous leukemia, chronic myelogenous leukemia, myelodysplastic syndrome and acute lymphoblastic leukemia. In a preferred embodiment, the disease is non-small cell lung cancer (NSCLC), breast cancer (e.g. hormone refractory metastatic breast cancer), head and neck cancer (e.g. head and neck squamous cell cancer), metastatic colorectal cancers, hormone sensitive or hormone refractory prostate cancer, colorectal cancer, ovarian cancer, hepatocellular cancer, renal cell cancer, soft tissue sarcoma, or small cell lung cancer.

In certain embodiments, methods disclosed herein further comprise administering a chemotherapeutic agent, targeted therapy, radiation, cryotherapy or hyperthermia therapy to the subject either prior to, simultaneously with, or after treatment with the combination therapy. In an additional aspect, the tumor may be resected prior to the administration of the checkpoint inhibitor.

### Examples

Experiments were performed using PNP with checkpoint inhibitor therapy in SC xenograft mouse models of colon and breast cancer to show additive or synergistic benefits. Results of the experiments indicate: 1) large breast tumors can be safely eradicated in syngeneic mice using a PNP-based approach; 2) expressing E. coli PNP in a solid breast tumor augments activity of checkpoint blockade inhibitors; 3) PNP based tumor treatment is synergistic or additive with checkpoint blockade type agents; 4) eradicating a PNP-expressing tumor has a strong abscopal effect (i.e. distant tumors not expressing PNP are more effectively treated by checkpoint blockade inhibitors when a tumor elsewhere in the body is eliminating using the PNP/fludarabine phosphate approach); and 5) fludarabine phosphate alone (a drug with tumor modulating properties) as a single agent enhances activity of checkpoint blockade inhibitors.

Effect of PNP Expression in Murine Breast Tumor Cells on Tumor Sensitivity to antiCTLA4-9H10.

In order to evaluate efficacy, the parental EMT-6-EMU cell line and the EMT-6-PNP-EMU transduced cell line were evaluated. The EMT-6-PNP-EMU transduced cell line and the parental EMT-6-EMU cell line were implanted as shown below in each flank with or without dosing with fludarabine phosphate and/or anti-CTLA-4 antibody using female BALB/c mice. Table 1 shows the experimental conditions.

| | | Formulation | Active | |
|---|---|---|---|---|
| Gr. | Agent | dose | dose | Schedule |
| 1^{#} | vehicle | | na | tid x 3 |
| 2 | fludarabine | 75 mg/kg | 75 mg/kg | tid x 3 |
| 3 | fludarabine | 90 mg/kg | 90 mg/kg | tid x 3 |
| 4 | anti-CTLA-4 9H10 // | 5 mg/kg // | 5 mg/kg // | day 1 // |
| | anti-CTLA-4 9H10 | 2.5 mg/kg | 2.5 mg/kg | days 4, 7 |
| 5 | fludarabine // | 75 mg/kg // | 75 mg/kg // | tid x 3 // |
| | anti-CTLA-4 9H10 // | 5 mg/kg // | 5 mg/kg // | day 1 // |
| | anti-CTLA-4 9H10 | 2.5 mg/kg | 2.5 mg/kg | days 4, 7 |
| 6 | fludarabine // | 90 mg/kg // | 90 mg/kg // | tid x 3 // |
| | anti-CTLA-4 9H10 // | 5 mg/kg // | 5 mg/kg // | day 1 // |
| | anti-CTLA-4 9H10 | 2.5 mg/kg | 2.5 mg/kg | days 4, 7 |
| 7 | vehicle | | na | tid x 3 |
| 8 | fludarabine | 90 mg/kg | 90 mg/kg | tid x 3 |
| 9 | anti-CTLA-4 9H10 // | 5 mg/kg // | 5 mg/kg // | day 1 // |
| | anti-CTLA-4 9H10 | 2.5 mg/kg | 2.5 mg/kg | days 4, 7 |
| 10 | fludarabine // | 90 mg/kg // | 90 mg/kg // | tid x 3 // |
| | anti-CTLA-4 9H10 // | 5 mg/kg // | 5 mg/kg // | day 1 // |
| | anti-CTLA-4 9H10 | 2.5 mg/kg | 2.5 mg/kg | days 4, 7 |

Mice were implanted with EMT-6 murine tumors on both the right and left flanks. In groups 1 through 6 the right flanks were implanted with parental (non-PNP) tumors and the left flanks were implanted with tumors expressing E. coli PNP. In groups 7 through 10, both the right and left flanks were injected with parental tumors.

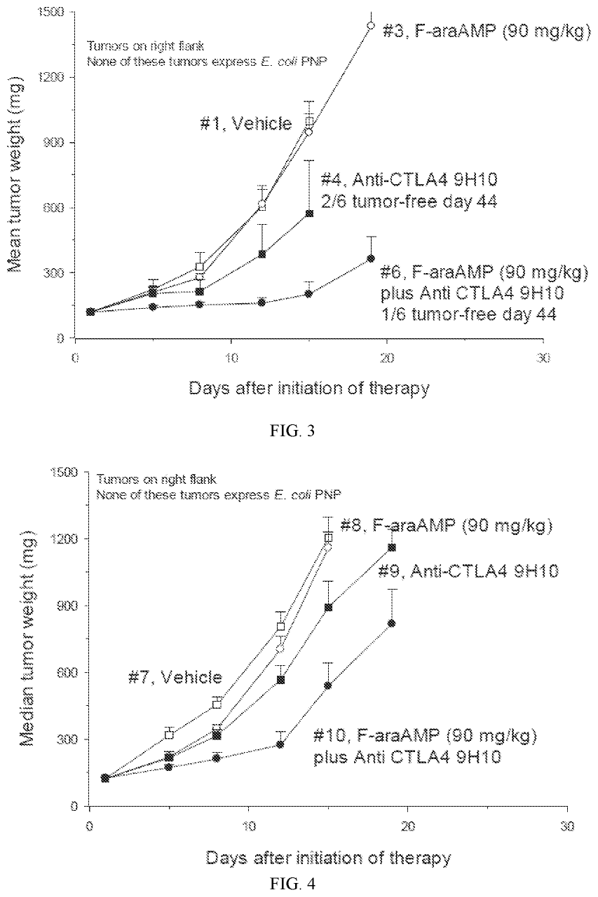

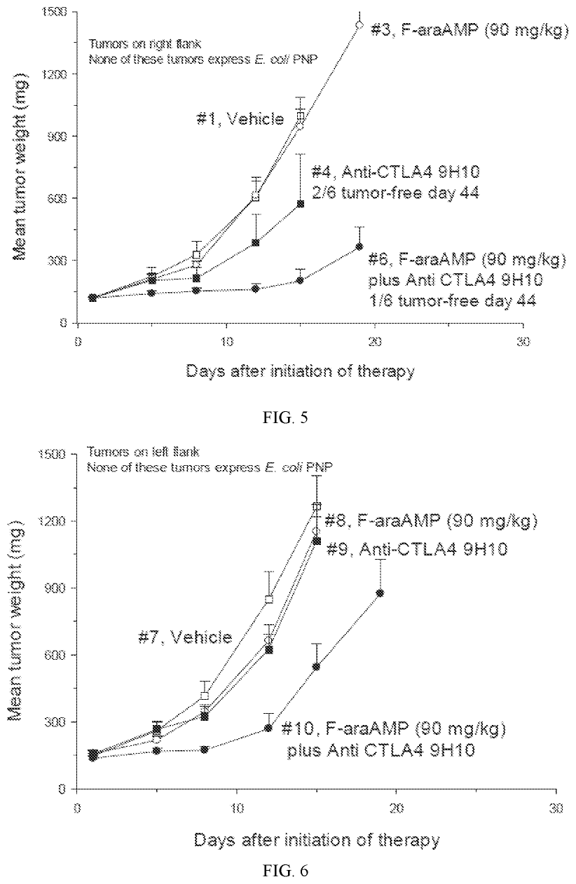

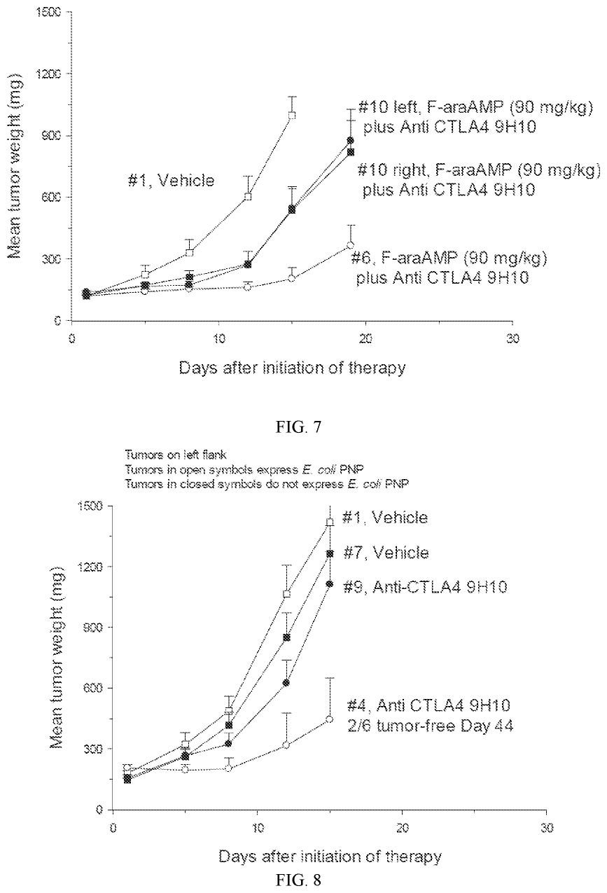

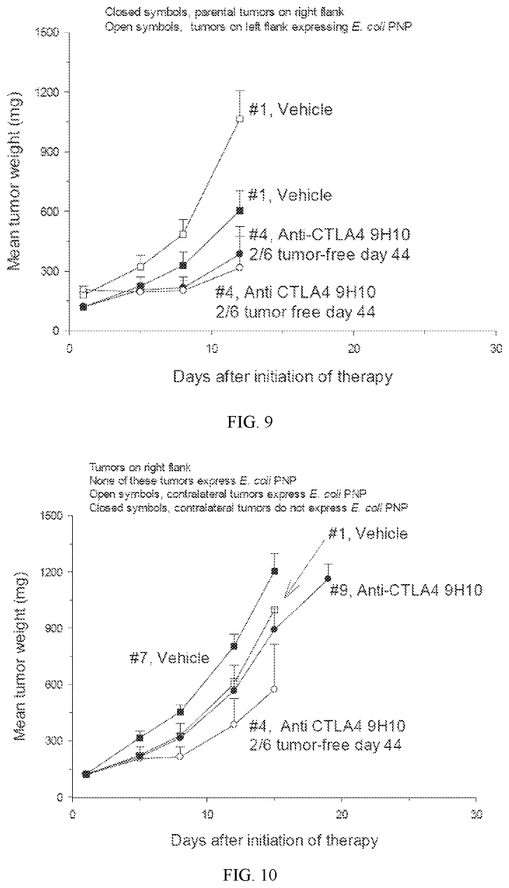

Experimental data indicates that PNP/fludarabine phosphate treatments improve treatments in combination with an anti-CTLA4 antibody. See figures, e.g., 1, 2, and 3. An abscopal effect was observes in which regression of a tumor expressing PNP and given fludarabine phosphate treatments also inhibits a non-PNP tumor elsewhere in the host animal. See, e.g., FIG. 4 and compare FIGS. 6 and 7 (group 6 right flank). Experiments also indicate fludarabine phosphate in the absence of PNP expression enhances the anticancer effects of anti-CTLA4 antibody. See, e.g., FIG. 6. It is also possible that PNP expression in the absence of prodrug enhances anti-CTLA4 antibody anti-cancer activity. See, e.g., FIGS. 8 and 9.

1. A method of treating cancer or initiating, enhancing, or prolonging an anti-tumor response in a subject in need thereof comprising administering to the subject an effective amount of a checkpoint inhibitor in combination with a non-mammalian purine nucleoside phosphorylase or nucleoside hydrolase or a vector encoding expression thereof, and a prodrug cleaved by said purine cleaving enzyme.

2. The method of claim 1 wherein administering to the subject a non-mammalian purine nucleoside phosphorylase or nucleoside hydrolase or a vector encoding expression thereof is a direct injection into replicating or non-replicating targeted cells and optionally exposure of the targeted cells to X-ray radiation.

3. The method of claim 2 wherein said replicating or non-replicating targeted cells are cancerous or define a tumor.

4. The method of claim 1 wherein said vector is viral vector.

5. The method of claim 1 wherein said non-mammalian purine nucleoside phosphorylase is derived from E. coli or T. vaginalis.

6. The method of claim 1 wherein said non-mammalian purine nucleoside phosphorylase is a mutant of E. coli purine nucleoside phosphorylase.

7. The method of claim 1 wherein said prodrug is 2-F-2′-deoxyadenosine (F-dAdo) or fludarabine phosphate (F-araAMP), derivative, or salt thereof.

8. The method of claim 1 wherein the checkpoint inhibitor is a biologic therapeutic or a small molecule.

9. The method of claim 1 wherein the checkpoint inhibitor is a monoclonal antibody, a humanized antibody, a fully human antibody, a fusion protein or a combination thereof.

10. The method of claim 1 wherein the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof.

11. The method of claim 1 wherein the checkpoint inhibitor is a PD-1, a PDL-1 and/or a CTLA-4 checkpoint inhibitor.

12. The method of claim 1 wherein the checkpoint inhibitor is selected from ipilimumab (anti-CTLA-4 antibody), nivolumab, pembrolizumab, and cemiplimab (anti-PD-1 antibodies), atezolizumab, durvalumab, and avelumab (anti-PD-L1 antibodies).

13. The method of claim 1 wherein the cancer is chronic lymphocytic leukemia (CLL).

14. The method of claim 1 wherein the cancer is breast cancer.

15. The method of claim 1 wherein the cancer is colon cancer.

16. A method of treating cancer or initiating, enhancing, or prolonging an anti-tumor response in a subject in need thereof comprising administering to the subject an effective amount of a checkpoint inhibitor in combination with a non-mammalian purine nucleoside phosphorylase or nucleoside hydrolase or a vector encoding expression thereof in the absence of a prodrug cleaved by said purine cleaving enzyme.

17. A method of treating cancer or initiating, enhancing, or prolonging an anti-tumor response in a subject in need thereof comprising administering to the subject an effective amount of a checkpoint inhibitor in combination with a prodrug.

18. The method of claim 17 wherein said prodrug is 2-F-2′-deoxyadenosine (F-dAdo) or fludarabine phosphate (F-araAMP), derivative, or salt thereof.
